(19) United States
(12) Patent Application Publication
(10) Pub. No.: US 2007/0282231 A1
(43) Pub. Date: Dec. 6, 2007

(54) Arm and shoulder support harness

(76) Inventors: Susan Picard, Poultney, VT (US); Janet Tulis, Norwell, MA (US)

Correspondence Address: COOK, ALEX, MCFARRON, MANZO, CUMMINGS & MEHLER LTD, SUITE 2850, 200 WEST ADAMS STREET, CHICAGO, IL, 60606, US

(21) Appl. No.: 11/446,690

(22) Filed: Jun. 5, 2006

(51) Int. Cl. A61F 5/00; A61F 5/37

(52) U.S. Cl. 602/4; 128/875

(57) ABSTRACT

A harness is provided for supporting an arm or an article against the front torso of a body. A fastener of the harness is fastened to clothing at the rear waistline and a strap extends upwardly from the fastener and over a shoulder of the body to the front torso. A support member or loop disposed at a free end of the strap adjacent to the front torso receives and supports an arm in a bent or flexed position against the front torso. A second strap, fastener, and support member provide enhanced support for the arm. Special application for the harness has been found for supporting an injured arm in a cast.

### FIELD OF THE INVENTION

This invention generally relates to a support harness wearable by a human subject. More particularly, this invention relates to a harness for supporting or immobilizing an arm or shoulder of a human subject.

### DESCRIPTION OF RELATED ART

Shoulder and arm injuries are common occurrences that are generally treated by surgery and subsequent immobilization. If the arm is not properly supported, then there is the risk of discomfort, pain, and an improper or unnecessarily lengthy healing process. If a bone is broken, then it is particularly important to properly position the arm in order to facilitate proper healing.

Typically, the patient will receive a splint or hard cast to prevent shifting of a broken bone or movement of the bones of the arm with respect to each other. Casts have two layers—a soft layer, usually comprised of cotton or a similar material, and a hard outer layer or shell. The outer layer is generally formed from either plaster of paris or fiberglass and can be relatively heavy, especially if the cast covers the entire arm.

The cast must remain on the arm for weeks or even months, so casts are often provided with a sling for additional support. A sling is a bandage that is placed under the arm and wrapped around the back of the neck. Well-known neck-supported slings include triangle slings and Cravat slings. One problem with such slings is that the weight of the arm and cast are supported by the neck, which can cause chronic discomfort and considerable pain, especially if the patient is older or has a history of back and/or neck problems.

Another problem with typical slings is that they include a continuous sleeve that surrounds the cast for supporting the arm. Such sleeves are uncomfortable, because they allow heat to build up in and around the cast. Furthermore, slings typically support the forearm in a relatively unadjustable horizontal position, which may allow for swelling in the hand and arm.

Accordingly, a general aspect or object of the present invention is to provide a harness for supporting an arm or shoulder.

Another aspect or object of this invention is to provide a harness for supporting an arm or shoulder without undue neck pain and discomfort.

Other aspects, objects and advantages of the present invention, including the various features used in various combinations, will be understood from the following description according to preferred embodiments of the present invention, taken in conjunction with the drawings in which certain specific features are shown.

### SUMMARY OF THE INVENTION

In accordance with one aspect of the present invention, a harness is provided for engaging a rear waistline of a patient. A fastener of the harness is fastened to the clothing of the patient, typically to a pair of pants or belt, but the fastener can be connected to anything wearable on, near or around the waist, so the term “clothing” is used broadly herein and includes items and accessories such as “fanny packs” and the like. A strap extends upwardly from the fastener and over a shoulder of the patient to the chest or front torso. A support member or loop disposed at a free end of the strap is sized and configured to receive and support a portion of an arm of the patient in a bent or flexed position against the chest or front torso. Preferably, the harness is similar to a pair of suspenders, with a pair of straps extending from the rear waistline of the patient, wherein one strap passes over each shoulder.

The free end of each strap includes a support member for receiving and supporting a portion of an arm in a bent or flexed position against the chest or front torso. The support member preferably takes the form of an extension of the strap shaped into a loop. Thus, it will be understood that the arm and a cast or splint, if provided, are effectively supported by the shoulders instead of the neck, thereby providing considerable relief for patients requiring long-term support or immobilization of an arm or shoulder. Furthermore, the harness prevents heat build-up, swelling, and other drawbacks associated with typical slings.

Modifications to the above-described harness include the addition of a swath or bandage to immobilize the arm against the chest or front torso. The same function can instead be provided by fasteners associated with the support members for securing the support members to the clothing of the patient. The straps and support members may be comprised of an elastic material for added comfort and may also be adjustable for achieving the proper length or loop size.

According to another aspect of the present invention, the harness may be used to support an article typically supported by a neck strap, such as a camera or pair of binoculars. When used to support such an article, the harness is provided with two support members, each having a fastener suitable for engagement with the article or with a strap associated with the article.

Special application for the present invention has been found for supporting an injured arm in a hard cast. However, the present invention is also applicable to supporting unwrapped arms for physical therapy and/or unwrapped arms that are healthy, objects normally supported by a neck strap, as well as any other object that would be advantageous to support against the chest or front torso. Therefore, it will be understood that the products described herein are not limited to particular medical, therapeutic, or rehabilitative devices or applications.

### BRIEF DESCRIPTION OF THE DRAWINGS

FIG. 1 is a front perspective view of a harness according to an aspect of the present invention;

FIG. 2 is a rear elevational view of a human body wearing the harness of FIG. 1;

FIG. 3 is a front elevational view of a human body wearing the harness of FIG. 1;

FIG. 4 is a side elevational view of a human body wearing the harness of FIG. 1;

FIG. 5 is a front perspective view of another embodiment of a harness according to the present invention;

FIG. 6 is a front elevational view of a human body wearing the harness of FIG. 5;

FIG. 7 is a side elevational view of a human body wearing a harness according to another embodiment; and

FIG. 8 is a front elevational view of a harness according to another aspect of the present invention.

### DESCRIPTION OF THE PREFERRED EMBODIMENTS

As required, detailed embodiments of the present invention are disclosed herein; however, it is to be understood that the disclosed embodiments are merely exemplary of the invention, which may be embodied in various forms. Therefore, specific details disclosed herein are not to be interpreted as limiting, but merely as a basis for the claims and as a representative basis for teaching one skilled in the art to variously employ the present invention in virtually any appropriate manner.

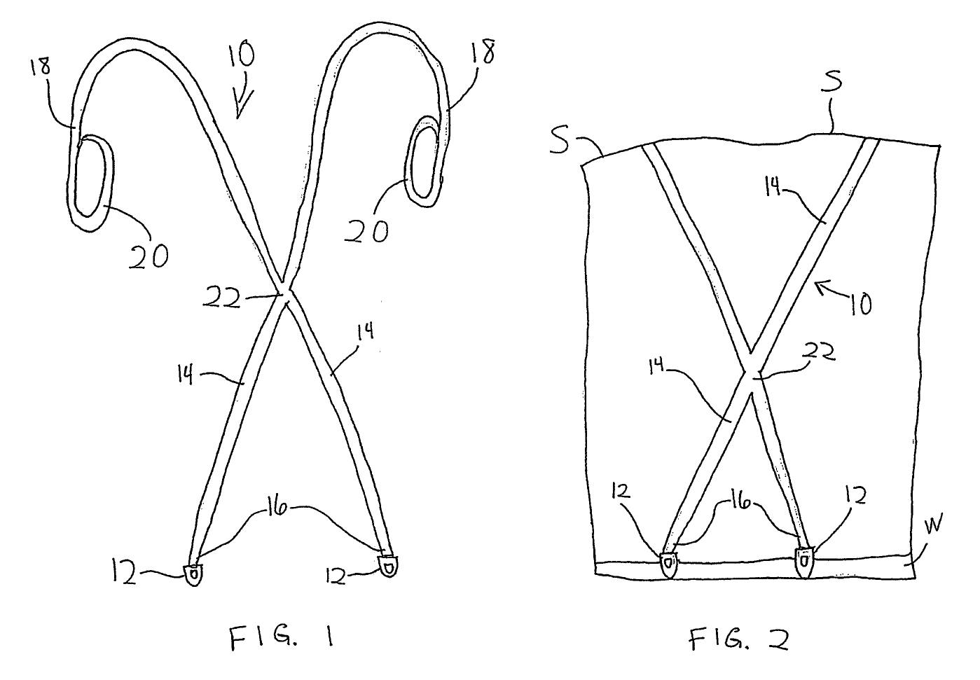

FIG. 1 illustrates a harness 10 according to an aspect of the present invention. The harness 10 includes a fastener 12 for securing the harness 10 to a rear waistline W of a patient, as illustrated in FIG. 2. When used herein, the term “fastener” refers to any means for securing the harness 10 to another structure, such clothing. The fastener 12 is to be secured to the clothing of a patient, so it can comprise a loop or hook for engaging a belt or a clamp for releasably gripping a pair of pants or a buttonhole for receiving a button or a strip of Velcro® or a buckle or a clasp or a clip or the like. Thus, it will be understood from the preceding non-exhaustive listing that the term “fastener” is not limiting, but is intended to cover any suitable fastener or joinder mechanism. Preferably, the fastener 12 is a compression fastener, such as disclosed in U.S. Pat. No. 4,463,482 to Hawie, which is hereby incorporated herein by reference. The illustrated harness 10 includes two identical fasteners 12, which are configured for engagement with different portions of the rear waistline W, as illustrated in FIG. 2. While it is possible to use a single fastener 12, it is preferable to provide a pair of fasteners 12 for distributing the weight of a supported arm over two shoulders of the body, as will be described herein.

Each fastener 12 includes a strap 14 extending generally upward in the configuration of FIGS. 1 and 2. Each strap 14 extends from a first anchored end 16 associated with a fastener 12 to a free end 18 associated with a loop or support member 20. The straps 14 are preferably formed of a relatively elastic or flexible material for improved comfort and adjustability. The straps 14 are also preferably between approximately 1.50 inches and 2.00 inches wide for improved weight distribution, comfort, and support.

As best illustrated in FIG. 4, each strap 14 extends from the rear waistline W of a patient, over a shoulder S, and to a front torso or chest region F. FIG. 2 shows a preferred embodiment in which the straps 14 cross each other, such that a strap fastened to a left portion of the rear waistline extends upwardly and over a right shoulder and a strap fastened to a right portion of the rear waistline extends upwardly and over a left shoulder. This configuration allows the straps 14 to be joined together at junction 22 and eliminates the risk of the straps 14 falling off of the shoulders S. Of course, the harness 10 may be practiced using two separate crossing or parallel straps 14, but it is preferable for straps 14 to be joined to form a unitary structure.

Each strap 14 preferably includes a clasp or buckle system or the like, not illustrated, for adjusting the length of the strap 14, which effectively varies the height of the associated support member 20 and, if two support members 20 are provided, the angle therebetween. This allows the harness to be used for people of different heights and differing arm orientation needs.

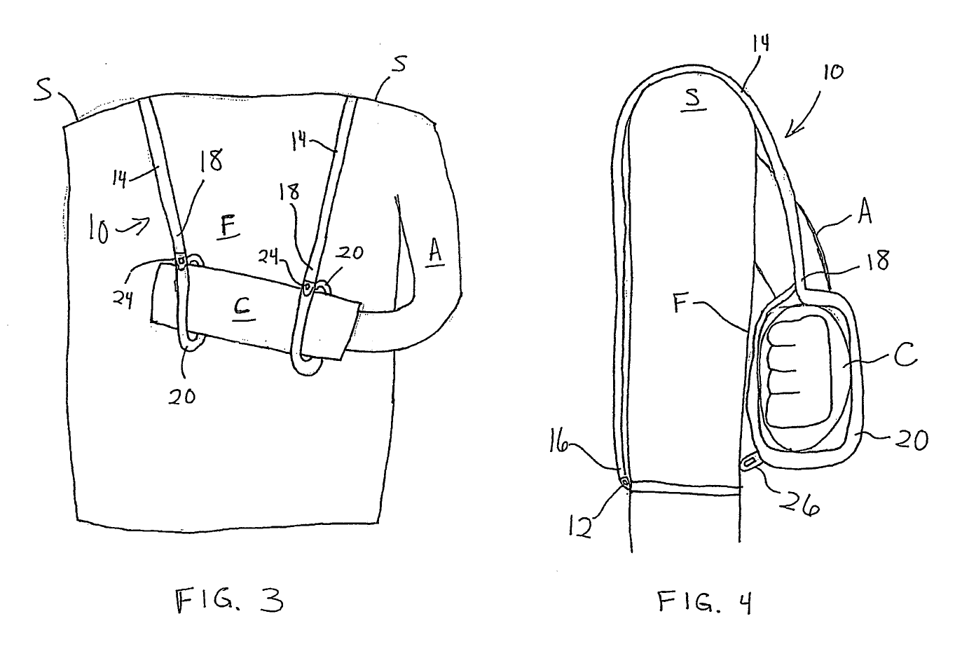

FIGS. 3 and 4 show that the free end 18 of each strap 14 includes a loop or support member 20 disposed at a front torso or chest region F. Each support member 20 is preferably an integral extension of the associated strap 14 and comprises a loop formed at the free end 18 of a strap 14 from the same elastic material, but each support member 20 could also be a separate structure which is connected to the free end 18 of the associated strap 14. For example, a loop or cuff may be sewed, adhered, or otherwise attached to the free end 18 of the strap 14 without departing from the scope of the present invention. If a separate support member is provided, then it preferably has a width approximately equal to the width of the strap 14 in order to provide optimal comfort and maneuverability. As illustrated, each support member 20 is sized and configured to receive and support a portion of an arm A in a bent or flexed position against the front torso F. The straps 14 may be adjusted to have different lengths, which may be practiced to prevent swelling or to make the patient more comfortable. It will be appreciated that more arm support angles are possible with a harness according to the present invention than a typical sling with a continuous sleeve.

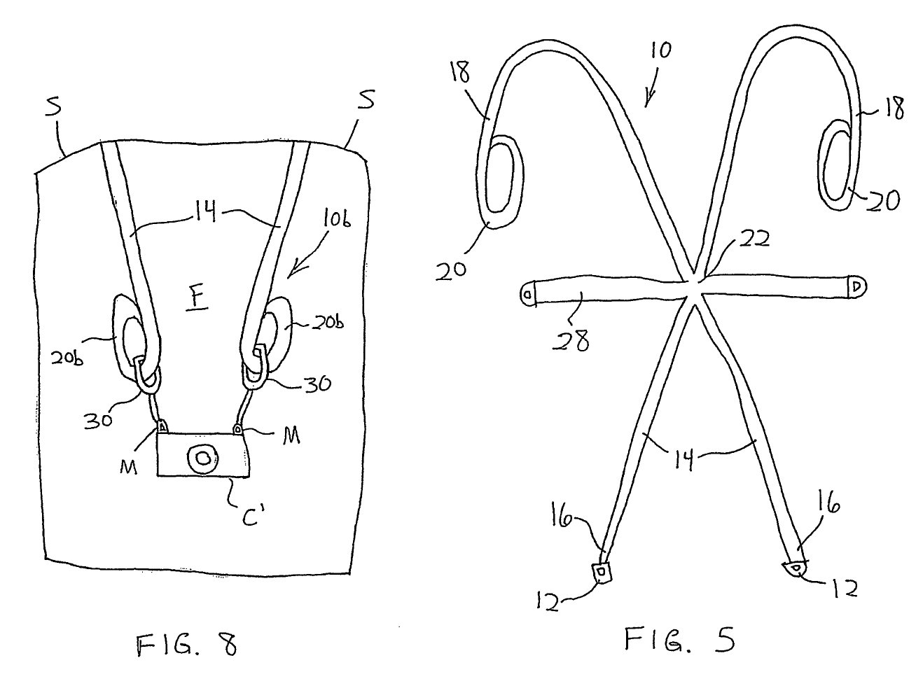

Each support member 20 is preferably provided with an adjustable clasp 24 or the like, for selectively adjusting the size of the support member 20. Additionally, each support member 20 may include a fastener 26 for securing the arm A to the clothing and front torso F of the patient, which prevents excessive movement and swinging of the arm A. Further immobilization of the arm A and shoulder S may be achieved by providing an elongated bandage or swath 28 connected to a strap 14 or the junction 22, as illustrated in FIGS. 5 and 6, for being wrapped around the arm A and front torso F to secure the arm A against the front torso F. Alternatively, the swath 28 may be passed between the arm A and the front torso F to pin the straps 14 against the front torso F for an effect similar to the fasteners 26 of FIG. 4. The swath 28 is preferably removably attached to the harness 10, so that it may be removed if not needed.

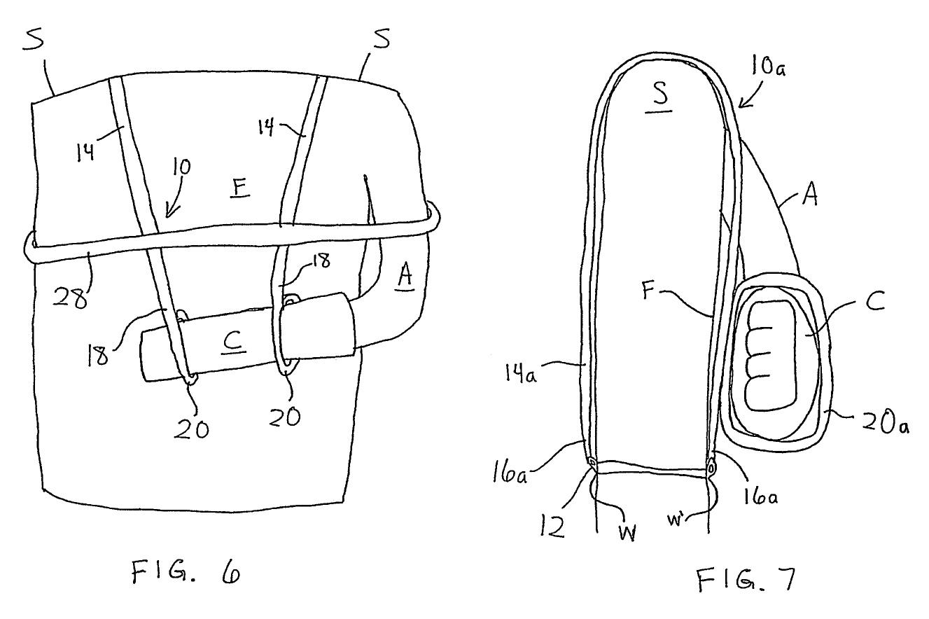

FIG. 7 shows yet another embodiment of a harness 10a according to the present invention. The harness 10a differs from the harness 10 of FIGS. 1-4 because both ends 16a of the strap 14a are anchored, one at the rear waistline W and the other at a front waistline W′, similar to a typical suspender strap. The strap 14a includes a supporting member or loop 20a that is permanently or removably attached to the strap 14a for receiving an arm A. Preferably, the supporting member 20a is comprised of the same elastic material as the strap 14a.

In addition to supporting an arm A or an arm A in a cast C, a harness according to the present invention can also be modified to support an article usually supported by a neck strap. For example, FIG. 8 illustrates a harness 10b supporting a camera C′. The camera C′ includes mounting fixtures or clips M adapted to receive a neck strap, not illustrated, to be wrapped behind the neck for support. A fastener 30 connected to each support member 20b effectively engages each mounting fixture M for supporting the camera C′. The harness 10b may be used alone or in combination with a neck strap, in order to provide additional security for heavy or expensive equipment. The strap 14 may be provided with a pocket or pouch, not illustrated, for holding a roll of film or the like.

It will be understood that the embodiments of the present invention which have been described are illustrative of some of the applications of the principles of the present invention. Numerous modifications may be made by those skilled in the art without departing from the true spirit and scope of the invention, including those combinations of features that are individually disclosed or claimed herein.

1. A harness wearable on a human body, comprising:
a fastener engageable with clothing located at a rear waistline of a human body;
a support member configured to receive and support at least a portion of an arm of the body, the arm being in a bent position adjacent to a front torso of the body; and
a strap extending between the fastener and the support member, said strap being configured to extend over a shoulder of the body.

2. The harness of claim 1, further comprising a further fastener engageable with clothing located at the rear waistline, an additional support member for receiving at least a portion of the arm, and a further strap extending between the further fastener and the additional support member, said further strap being configured to extend over another shoulder of the body.

3. The harness of claim 2, further comprising a junction joining the strap and the second strap together, said junction being spaced away from the fastener and from the further fastener and between said fasteners and the shoulders.

4. The harness of claim 1, wherein said support member has a width approximately equal to the width of the strap.

5. The harness of claim 1, wherein said strap is comprised of an elastic material.

6. The harness of claim 1, wherein said support member is a loop comprising an integral extension of the strap.

7. The harness of claim 1, wherein the length of the strap is adjustable.

8. The harness of claim 1, further comprising an adjustable clasp for adjusting the size of the supporting member.

9. The harness of claim 1, further comprising a swath associated with the strap and wrappable around the front torso and the arm for securing the arm to the front torso.

10. The harness of claim 1, further comprising a front fastener associated with said strap for connecting said strap to a front waistline of the body.

11. The harness of claim 1, further comprising a front fastener associated with the support member, the front fastener being for securing the support member to the front torso.

12. A harness wearable on a human body, comprising:
a support member configured to receive and support at least a portion of an arm of the body, the arm being in a bent position adjacent to a front torso of the body;
a strap associated with the support member and configured to extend over a shoulder of the body to an anchored end;
a fastener associated with the anchored end; and
an article of clothing at a rear waistline of the body, wherein said article of clothing is adapted to receive the fastener.

13. The harness of claim 12, further comprising a further fastener, an additional support member, and a further strap, wherein said article of clothing is adapted to receive the further fastener.

14. The harness of claim 12, wherein said strap is comprised of an elastic material.

15. The harness of claim 12, wherein said support member is a loop comprising an integral extension of the strap.

16. The harness of claim 12, wherein the length of the strap is adjustable.

17. The harness of claim 12, wherein the size of the supporting member is adjustable.

18. A harness wearable on a human body, comprising:
a first strap extending between a first fastener engageable with clothing at a rear waistline of the body and a first support member disposed adjacent to a front torso of the body, said first strap being configured to extend over a shoulder of the body;
a second strap extending between a second fastener engageable with clothing at a rear waistline of the body and a second support member disposed adjacent to a front torso of the body, said second strap being configured to extend over a second shoulder of the body;
a third fastener associated with the first support member; and
a fourth fastener associated with the second support member, wherein said third and fourth fasteners are configured for engagement with mounting fixtures of an article for supporting the article.

19. The harness of claim 18, wherein each support member is a loop comprising an integral extension of the associated strap.

20. The harness of claim 18, wherein the length of the straps is adjustable.
